(19) United States
(12) Patent Application Publication
(10) Pub. No.: US 2007/0212690 A1
(43) Pub. Date: Sep. 13, 2007

(54) Folate Antagonists Having Improved Selectivity

(75) Inventor: Jeffrey M. Partridge, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Serenex, Inc., Durham, NC (US)

(21) Appl. No.: 10/577,570

(22) PCT Filed: Oct. 28, 2004
(86) PCT No.: PCT/US04/35719

§ 371 Date: Feb. 22, 2007

Related U.S. Application Data
(60) Provisional application No. 60/515,012, filed on Oct. 28, 2003.

(51) Int. Cl. C12Q 1/68; C12Q 1/26

(52) U.S. Cl. 435/6; 435/25

(57) ABSTRACT

The present invention provides methods for identifying improved folate antagonists. The improved folate antagonists identified by the methods of the invention have increased selectivity. The increased selectivity of the folate antagonists results in a reduced risk of adverse effects following treatment with the improved folate antagonists. The improved folate antagonists are identified based on their reduced binding affinity for at least one enzyme selected from glutathione synthase, pyruvate carboxylase, propionyl-CoA carboxylase, biotin carboxylase, acetyl-CoA carboxylase, and methylcrotonyl-CoA carboxylase. According to the invention, folate antagonists having reduced affinity for at least one enzyme selected from this group of enzymes are selected for the treatment of neoplastic, hyperproliferative, and immune disorders.

### FIELD OF THE INVENTION

The present invention relates generally to drug discovery and more specifically to methods of selecting or identifying folate antagonists having improved selectivity.

### BACKGROUND OF THE INVENTION

Folate antagonists, including methotrexate (MTX), were first developed for the treatment of cancer and have subsequently been used to treat both neoplastic and non-neoplastic diseases. Methotrexate inhibits the folate-dependent enzyme dihydrofolate reductase (DHFR), and has also been shown to directly inhibit the activity of thymidylate synthase (TS) and phosphoribosylglycinamide formyltransferase (GART) (Purcell and Ettinger (2003) Current Oncology Reports 5:114-125).

MTX has been used in the treatment of a number of types of cancer, including lymphoblastic leukemia, meningeal leukemia, lymphoma, choriocarcinoma, osteosarcoma, mycosis fungoides, Burkitt's and other non-Hodgkins' lymphomas, and carcinomas of the breast, hand, neck, ovary, and bladder. In addition to its use in cancer chemotherapy, MTX is a potent inhibitor of cell-mediated immune reactions and has been used as an immunosuppressive agent. MTX is currently among the most commonly used treatments for rheumatoid arthritis (Weinblatt et al. (1985) N. Eng. J. Med. 312:818-322; and Williams et al. (1985) Arthritis Rheum. 28:721-730), and is also used to treat other chronic inflammatory disorders. MTX is also effective in the prophylaxis of acute graft-versus-host disease either alone or in association with cyclosporin A and/or prednisone (Storb et al. (1986) N. Engl. J. Med. 314:729-35; Nash et al. (1992) Blood 80:1838-45; and Chao et al. (1993) N. Engl. J. Med. 329:1225-30) or FK506 (Nash et al. (1996) Blood 88:3634-3641), and is used in an adjunct therapy for persistent mild cardiac allograft rejection (Olsen et al. (1990) Transplantation 50:773-75). Other immune disorders in which MTX is used include dermatomyositis, rheumatoid arthritis (Hoffmeister (1983) Am. J. Med. 30:69-73), Wegener's granulomatosis, Crohn's disease (Feagan et al. (1995) N. Eng. J. Med. 332:292-7), and multiple sclerosis and associated disorders of the central nervous system (U.S. Patent Application No. 20030008875). MTX has also been used to treat the abnormally rapid proliferation of epidermal cells associated with psoriasis (McDonald (1981) Pharmacol. Ther. 14:1-24).

Other folate antagonists are currently being developed for the treatment of neoplastic, hyperproliferative, and immune disorders. Examples include the DHFR inhibitors trimetrexate and edatrexate; the TS inhibitors raltitrexed, premetrexed, GW1843, OSI-7904L, nolatrexed, and ZD9331; and the GART inhibitors lomotrexol, and LY309887 (Purcell and Ettinger (2003) Current Oncology Reports 4:114-25).

MTX is the folate antagonist that is most commonly used in the treatment of neoplastic, hyperproliferative, and immune disorders. However a number of adverse effects are associated with MTX treatment, particularly when higher doses of MTX are used. These adverse effects include myelosuppression, alopecia, dermatitis, interstitial pneumonitis, nephrotoxicity, defective oogenesis or spermatogenesis, abortion, and teratogenesis. Hepatic dysfunction, usually reversible but sometimes leading to cirrhosis, also occurs in some cases. Intrathecal administration of MTX can cause meningismus and an inflammatory response in the cerebrospinal fluid. Seizures, coma, and death result in rare instances. Similarly, adverse effects observed with the folate antagonists trimetrexate, edatrexate, raltitrexed, premetrexed, GW1843, OSI-7904L, nolatrexed, ZD9331, lomotrexol, and LY309887 include myelosuppression, rash, mucositis, fever, diarrhea, nausea, vomiting, transaminitis, leukopenia, neutropenia, and thrombocytopenia.

Accordingly, there remains a need for methods of identifying folate antagonists having increased selectivity and decreased incidence of adverse effects.

### SUMMARY OF THE INVENTION

The present invention provides methods for identifying improved folate antagonists. The improved folate antagonists identified by the methods of the invention have increased selectivity. The increased selectivity of the folate antagonists results in a reduced risk of adverse effects following treatment with the improved folate antagonists. The improved folate antagonists are identified based on their reduced binding affinity for at least one enzyme selected from glutathione synthase, pyruvate carboxylase, propionyl-CoA carboxylase (PCCA and PCCB), biotin carboxylase (ACC1 and ACC2), acetyl-CoA carboxylase (COA1 and COA2) and methylcrotonyl-CoA carboxylase (MCCA and MCCB). According to the invention, folate antagonists having reduced affinity for at least one enzyme selected from this group of enzymes are selected for the treatment of neoplastic, hyperproliferative, and immune disorders.

Improved folate antagonists identified according to the method of the invention are encompassed. Also provided are methods of predicting whether a subject will have an increased risk of an adverse side effect during treatment with a folate antagonist. The methods rely on the finding that glutathione synthase, pyruvate carboxylase, PCCA, PCCB, ACC1, ACC2, COA1, COA2, MCCA, and MCCB are biomarkers for individuals having an increased risk of folate antagonist-induced adverse effects.

In one embodiment, the present invention provides a method of screening compounds to identify a folate antagonist having increased selectivity. The method comprises screening compounds to identify one or more compounds that act as folate antagonists and screening the identified folate antagonists to determine the level of binding to at least one enzyme selected from glutathione synthase, pyruvate caxboxylase, propionyl-CoA carboxylase, biotin carboxylase, acetyl-CoA carboxylase, and methylcrotonyl-CoA carboxylase, where a folate antagonist that has a low level of binding to at least one of these enzymes is identified as a folate antagonist having increased selectivity.

In another embodiment, the present method provides a method of screening folate antagonists to select the folate antagonists having the greatest selectivity. The method comprises screening two or more folate antagonists to determine their level of binding to at least one enzyme selected from glutathione synthase, pyruvate carboxylase, propionyl-CoA carboxylase, biotin carboxylase, acetyl-CoA carboxylase, and methylcrotonyl-CoA carboxylase, and selecting the folate antagonist that has the lowest level of binding to at least one enzyme selected from glutathione synthase, pyruvate carboxylase, propionyl-CoA carboxylase, biotin carboxylase, acetyl-CoA carboxylase, and methylcrotonyl-CoA carboxylase.

In yet another embodiment, the present invention provides a method of selecting a folate antagonist for use in therapy for a neoplastic, hyperproliferative, or immune disorder. The method comprises screening two or more folate antagonists to determine their level of binding to at least one enzyme selected from glutathione synthase, pyruvate carboxylase, propionyl-CoA carboxylase, biotin carboxylase, acetyl-CoA carboxylase, and methylcrotonyl-CoA carboxylase, and selecting the folate antagonist that has the lowest level of binding to at least one enzyme selected from glutathione synthase, pyruvate carboxylase, propionyl-CoA carboxylase, biotin carboxylase, acetyl-CoA carboxylase, and methylcrotonyl-CoA carboxylase as the folate antagonist for use in therapy for a neoplastic, hyperproliferative, or immune disorder.

The invention also provides a method of identifying a folate antagonist having a reduced risk of causing an adverse effect when it is used for treatment of a subject. The method comprises screening two or more folate antagonists to determine their level of binding to at least one enzyme selected from glutathione synthase, pyruvate carboxylase, propionyl-CoA carboxylase, biotin carboxylase, acetyl-CoA carboxylase, and methylcrotonyl-CoA carboxylase, and selecting the folate antagonist that has the lowest level of binding to at least one of these enzymes as the folate antagonist having a reduced risk of causing an adverse effect when it is used for treatment of a subject.

In some embodiments of the methods of the invention, the level of binding of the folate antagonist to at least one enzyme selected from glutathione synthase, pyruvate carboxylase, propionyl-CoA carboxylase, biotin carboxylase, acetyl-CoA carboxylase, and methylcrotonyl-CoA carboxylase is determined by directly measuring the binding affinity of the inhibitor for the enzyme.

In other embodiments of the invention, the level of binding of the folate antagonist to at least one enzyme selected from glutathione synthase, pyruvate carboxylase, propionyl-CoA carboxylase, biotin carboxylase, acetyl-CoA carboxylase, and methylcrotonyl-CoA carboxylase is determined by measuring the activity of the enzyme in the presence of the folate antagonist.

The level of glutathione synthase, pyruvate carboxylase, propionyl-CoA carboxylase, biotin carboxylase, acetyl-CoA carboxylase, and methylcrotonyl-CoA carboxylase may be measured, for example, in a cell-based assay or a cell-free assay.

The invention also encompasses folate antagonists identified or selected according to the methods of the invention.

In a further embodiment, the invention provides a method for determining the selectivity of a folate antagonist for a folate-dependent enzyme and for predicting whether a folate antagonist will have adverse effects when used to treat a subject. The methods comprise determining the level of binding of the folate antagonist to at least one enzyme selected from glutathione synthase, pyruvate carboxylase, propionyl-CoA carboxylase, biotin carboxylase, acetyl-CoA carboxylase, and methylcrotonyl-CoA carboxylase.

The invention provides improvements in a method of screening for folate antagonists for use in the treatment of neoplastic, hyperproliferative, or immune disorders. The improvement comprises determining the level of binding of one or more folate antagonists to at least one enzyme selected from glutathione synthase, pyruvate carboxylase, propionyl-CoA carboxylase, biotin carboxylase, acetyl-CoA carboxylase, and methylcrotonyl-CoA carboxylase.

Also provided is an improvement in a method of screening for folate antagonists having increased selectivity. The improvement comprises determining the level of binding of one or more folate antagonists to at least one enzyme selected from glutathione synthase, pyruvate carboxylase, propionyl-CoA carboxylase, biotin carboxylase, acetyl-CoA carboxylase, and methylcrotonyl-CoA carboxylase.

In another embodiment, the invention provides a method of screening for drugs that may be used to improve treatment of neoplastic, hyperproliferative, and immune disorders. The method comprises identifying one or more compounds that inhibit the activity of a folate-dependent enzyme and screening these compounds to determine their level of binding to at least one enzyme selected from glutathione synthase, pyruvate carboxylase, propionyl-CoA carboxylase, biotin carboxylase, acetyl-CoA carboxylase, and methylcrotonyl-CoA carboxylase. According to the method, a compound that has a low level of binding to at least one enzyme selected from glutathione synthase, pyruvate carboxylase, propionyl-CoA carboxylase, biotin carboxylase, acetyl-CoA carboxylase, and methylcrotonyl-CoA carboxylase is identified as a drug that may be used to improve treatment of neoplastic, hyperproliferative, and immune disorders.

The invention further provides an improvement in a method of treating a neoplastic disorder, hyperproliferative disorder, or immune disorder in a subject. The improvement comprises treating the subject with a folate antagonist identified according to the screening methods of the invention.

The invention also provides a method of screening subjects to identify those having an increased risk of developing adverse effects following treatment with a folate antagonist. In some embodiments, the method comprises determining the level of at least one enzyme selected from glutathione synthase, pyruvate carboxylase, propionyl-CoA carboxylase, biotin carboxylase, acetyl-CoA carboxylase, and methylcrotonyl-CoA carboxylase in a sample from the subject and comparing this level with a range of values representing the levels of the same enzyme or enzymes in subjects who have developed adverse effects following treatment with a folate antagonist. A subject having a level of at least one enzyme selected from glutathione synthase, pyruvate carboxylase, propionyl-CoA carboxylase, biotin carboxylase, acetyl-CoA carboxylase, and methylcrotonyl-CoA carboxylase falling within the range of values for the levels of the same enzyme or enzymes in subject who have experienced an adverse effect as a result of treatment with a folate antagonist is identified as a subject having an increased risk of developing an adverse reaction following treatment with a folate antagonist.

In other embodiments, the method of screening subjects to identify those having an increased risk of developing adverse effects following treatment with a folate antagonist, the level of at least one enzyme selected from glutathione synthase, pyruvate carboxylase, propionyl-CoA carboxylase, biotin carboxylase, acetyl-CoA carboxylase, and methylcrotonyl-CoA carboxylase in a sample from the subject is compared with a range of values representing the levels of the same enzyme or enzymes in normal subjects. A subject having a level of at least one enzyme selected from glutathione synthase, pyruvate carboxylase, propionyl-CoA carboxylase, biotin carboxylase, acetyl-CoA carboxylase, and methylcrotonyl-CoA carboxylase that is significantly lower than the level in normal subjects is identified as a subject having an increased risk of developing an adverse reaction following treatment with a folate antagonist.

The invention further provides a kit for use in a method of screening subjects to identify those having an increased risk of developing an adverse effect following treatment with a folate antagonist. The kit includes reagents for use in determining a value representing the level of at least one enzyme selected from glutathione synthase, pyruvate carboxylase, propionyl-CoA carboxylase, biotin carboxylase, acetyl-CoA carboxylase, and methylcrotonyl-CoA carboxylase in the subject and instructions for use in a method of determining whether the subject has an increased risk of developing adverse effects following treatment with a folate antagonist.

The invention also encompasses a method of selecting a therapy for a subject affected by a neoplastic, hyperproliferative, or immune disorder. In some embodiments, the method involves determining a value representing the level of at least one enzyme selected from glutathione synthase, pyruvate carboxylase, propionyl-CoA carboxylase, biotin carboxylase, acetyl-CoA carboxylase, and methylcrotonyl-CoA carboxylase by analyzing a sample taken from the subject, determining whether this value falls within a range of values representing the levels of the same enzyme or enzymes in subjects who have developed adverse effects as a result of treatment with a folate antagonist; and selecting a therapy for the patient based on the results of the comparison. When the value representing the level of glutathione synthase, pyruvate carboxylase, propionyl-CoA carboxylase, biotin carboxylase, acetyl-CoA carboxylase, and methylcrotonyl-CoA carboxylase in the subject falls within the range of values representing the levels of these enzymes in subjects who have experienced an adverse effect as result of treatment with a folate antagonist, a therapy that decreases the risk of the adverse effects is selected.

In other embodiments, the method of selecting a therapy for a subject affected by a neoplastic, hyperproliferative, or immune disorder, the level of at least one enzyme selected from glutathione synthase, pyruvate carboxylase, propionyl-CoA carboxylase, biotin carboxylase, acetyl-CoA carboxylase, and methylcrotonyl-CoA carboxylase in a sample from the subject is compared with a range of values representing the levels of the same enzyme or enzymes in normal subjects. When the value representing the level of glutathione synthase, pyruvate carboxylase, propionyl-CoA carboxylase, biotin carboxylase, acetyl-CoA carboxylase, and methylcrotonyl-CoA carboxylase in the subject is significantly lower that that for normal subjects, a therapy that decreases the risk of the adverse effects is selected.

### DESCRIPTION OF THE INVENTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention provides methods and compositions for the improved treatment of neoplastic, hyperproliferative, and immune disorders. The methods of the invention include methods for identifying improved folate antagonists for the treatment of neoplastic, hyperproliferative, and immune disorders. The improved folate antagonists identified by the methods of the invention have increased selectivity. The increased selectivity of these folate antagonists results in a reduced risk of adverse effects following treatment with the improved folate antagonists. The improved folate antagonists are identified based on their reduced binding affinity for one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase. According to the invention, folate antagonists having reduced affinity for one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase are selected for the treatment of neoplastic, hyperproliferative, and immune disorders. The invention also encompasses folate antagonists identified according to the screening methods of the invention, and methods of predicting whether a subject will have an increased risk of an adverse side effect as a result of treatment with a folate antagonist.

The present invention identifies six new target enzymes for the folate antagonist methotrexate (MTX). It is the novel finding of the present invention that MTX binds to the enzymes glutathione synthase, pyruvate carboxylase, propionyl-CoA carboxylase, biotin carboxylase, acetyl-CoA carboxylase, and methylcrotonyl-CoA carboxylase, in addition to the previously-identified targets dihydrofolate reductase, thymidylate synthase, and phosphoribosylglycinamide formyltransferase (GART).

Glutathione synthase (EC 6.3.2.3) catalyzes the second step in glutathione synthesis:
ATP+γ-L-glutamyl-L-cysteine+glycine<=>ADP+phosphate+glutathione
Glutathione is required for a variety of biologic functions, including protection of cells from oxidative damage by free radicals, detoxification of xenobiotics, and membrane transport (Meister and Anderson (1983) Annu. Rev. Biochem. 52:711-60; and Uhlig and Wendel (1992) Life Sci. 51:1083-94). The human amino acid sequence for glutathione synthase is described by Gali and Board (1995) Biochem. J. 310: 353-58 and is given in Swiss-Prot accession number P48637, both of which are herein incorporated by reference. Particular mutations in the human glutathione synthase gene cause 5-oxoprolinuria (pyroglutamicaciduria), which, in its severe form, is characterized by massive urinary secretion of 5-oxoproline, metabolic acidosis, hemolytic anemia, and central nervous system damage (Shi et al. (1996) Nature Genet. 14:361-65; Dahl et al. (1997) Hum. Molec. Genet. 6:1147-52). By “glutathione synthase” as used herein, it is intended an enzyme or enzyme subunit from enzyme class 6.3.2.3. In particular embodiments, the glutathione synthase is mammalian glutathione synthase, such as, for example human glutathione synthase, although the glutathione synthase may be from any source.

Pyruvate carboxylase (EC 6.4.1.1) catalyzes the ATP-dependent carboxylation of pyruvate:
ATP+pyruvate+HCO₃⁻<=>ADP+phosphate+oxaloacetate
Pyruvate carboxylase is a key regulatory enzyme in gluconeogenesis, lipogenesis, and neurotransmitter synthesis. The human amino acid sequence for pyruvate carboxylase is described by Freytag and Collier (1984) J. Biol. Chem. 259:12831-37 and is given in Swiss-Prot accession number JC2460, both of which are herein incorporated by reference. Two distinct clinical presentations of pyruvate deficiency have been identified. An infantile form present soon after birth with chronic lacticacidemia, and delayed neurologic development in survivors. The second form also presents early with lacetic acidosis but shows elevated blood levels of ammonia, citrulline, proline, and lysine. Mutations in the human pyruvate carboxylase gene that result in pyruvate carboxylase deficiency have been identified (Carbone et al. (1998) Am. J. Hum. Genet. 62:1312-19; Wexler et al. (1998) Pediat. Res. 43:579-84; and Carbone et al. (2002) Hum. Mutat. 20:48-56). By “pyruvate carboxylase” as used herein, it is intended an enzyme or enzyme subunit from enzyme class 6.4.1.1. In particular embodiments, the pyruvate carboxylase is mammalian pyruvate carboxylase, such as, for example human pyruvate carboxylase, although the pyruvate carboxylase may be from any source.

Propionyl-CoA carboxylase (EC 6.4.1.3) catalyzes the first step in the catabolism of propionyl-CoA:
ATP+propanoyl-CoA+HCO₃⁻<=>ADP+phosphate+(S)-methylmalonyl-CoA
This enzyme also carboxylates butanoyl-CoA and catalyzes transcarboxylation. Propionyl-CoA is an important intermediate in the metabolism of isoleucine, threonine, methionine, and valine. Propionyl-CoA carboxylase is composed of two non-identical subunits, alpha (PCCA) and beta (PCCB). The human amino acid sequences for PCCA and PCCB are described by Lamhonwah et al. (1986) Proc. Natl. Acad. Sci. U.S.A. 83:4864-4868 and are given in Swiss-Prot accession numbers P05165 (PCCA) and P05166 (PCCB), each of which is herein incorporated by reference. Mutations in the human PCCA and PCCB gene are responsible for propionic acidemia, an autosomal recessive disease characterized by an excess of propionic acid in the blood and urine, with ketosis, acidosis, hyperglycinemia, hyperglycinuria, and often neurologic complications (Lamhonwah et al. (1986) Proc. Natl. Acad. Sci. U.S.A. 83:4864-4868). By “propionyl-CoA carboxylase” as used herein, it is intended an enzyme or enzyme subunit from enzyme class 6.4.1.3, or a subunit of such an enzyme (e.g. PCCA or PCCB). In particular embodiments, the propionyl-CoA carboxylase is mammalian propionyl-CoA carboxylase, such as, for example human propionyl-CoA carboxylase, although the propionyl-CoA carboxylase may be from any source.

Acetyl-CoA carboxylase (EC 6.4.1.2) catalyzes the following reaction:
ATP+acetyl-CoA+HCO₃⁻<=>ADP+phosphate+malonyl-CoA
Malonyl-CoA is the C₂ donor in the de novo synthesis of long chain fatty acids and in their elongation into very long chain fatty acids. At least two different human proteins having acetyl-CoA carboxylase activity have been identified. The first, COA1, was described in Abu-Elheiga et al. (1995) Proc. Natl. Acad. Sci. USA 92:4011-15, and its amino acid sequence is given in Swiss-Prot accession number Q13085, both of which are herein incorporated by reference. COA1 is primarily cytoplasmic and catalyzes the rate-limiting step in the biogenesis of long-chain fatty acids. The second protein, COA2, was described in Abu-Elheiga et al. (1997) J. Biol. Chem. 272:10669-77, and its amino acid sequence is given in Swiss-Prot accession number 000763, both of which are herein incorporated in their entirety. COA2 is involved in the regulation of fatty acid oxidation. Deficiency in acetyl-CoA carboxylase activity causes hypotonic myopathy and neurologic damage (Blom et al. (1981) Eng. J. Med. 305:465-66). Mice with a targeted disruption in COA2 demonstrated a higher fatty acid oxidation rate, as well as 10-fold lower levels of malonyl CoA in heart and 30-fold lower levels of malonyl CoA in muscle (Abu-Elheiga et al. (2001) Science 291:2613-16). By “acetyl-CoA carboxylase” as used herein, it is intended an enzyme or enzyme subunit from enzyme class EC 6.4.1.2. In particular embodiments, the acetyl-CoA carboxylase is a mammalian acetyl-CoA carboxylase, such as, for example human acetyl-CoA carboxylase, although the acetyl-CoA carboxylase may be from any source.

Biotin carboxylase (EC 6.3.4.14) catalyzes the ATP-dependent carboxylation of biotin:
ATP+biotin-carboxyl carrier protein+HCO₃⁻<=>ADP+phosphate+carboxybiotin-carboxyl-carrier-protein
This enzymatic activity is required for the synthesis of long chain fatty acids. The two human acetyl-CoA proteins COA1 (ACC1) and COA2 (ACC2) described above also have biotin carboxylase activity. By “biotin carboxylase” as used herein, it is intended an enzyme or enzyme subunit from enzyme class 6.3.4.14. In particular embodiments, the biotin carboxylase is a mammalian biotin carboxylase, such as, for example human biotin carboxylase, although the biotin carboxylase may be from any source.

Methylcrotonyl carboxylase (EC 6.4.1.4) catalyzes the following reaction:
ATP+3-methylcrotonyl-CoA+HCO₃⁻<=>ADP+phosphate+3-methylglutaconyl-CoA
Methylcrotonyl carboxylase is involved in the breakdown of the branched chain amino acid leucine. This enzyme is a heteromeric protein containing alpha and beta subunits (MCCA and MCCB, respectively). Human MCCA was described by Gallardo et al. (2001) Am J. Hum. Genet. 68:334-346, and the amino acid sequence of this protein is given in Swiss-Prot accession number Q96RQ3, both of which are herein incorporated by reference. MCCB was also described in Gallardo et al. (2001) Am J. Hum. Genet. 68:334-346, and its amino acid sequence is given in Swiss-Prot accession number Q9HCC0, both of which are herein incorporated in their entirety. Mutations in either MCCA or MCCB can cause 3-methylcrotonylglycinuria, a defect in leucine catabolism. This disorder is characterized by urinary excretion of 3-hydroxyisovaleric acid and 3-methylcrotonylglycine. The clinical phenotypes of 3-methylcrotonylglycinuria are highly variable, and range from asymptomatic to profound metabolic acidosis. By “methylcrotonyl carboxylase” as used herein, it is intended an enzyme or enzyme subunit from enzyme class EC 6.4.1.4. In particular embodiments, the methylcrotonyl carboxylase is a mammalian acetyl-CoA carboxylase, such as, for example human methylcrotonyl carboxylase, although the methylcrotonyl carboxylase may be from any source.

The use of MTX in therapy for neoplastic, hyperproliferative, and immune disorders is associated with a number of adverse effects, particularly when MTX is used a higher dosages. MTX-associated adverse effects include alopecia, dermatitis, interstitial pneumonitis, nephrotoxicity, defective oogenesis or spermatogenesis, abortion, and teratogenesis, hepatotoxicity, hepatic dysfunction, cirrhosis, and damage to the central nervous system.

The identification of glutathione synthase, pyruvate carboxylase, propionyl-CoA carboxylase, biotin carboxylase, acetyl-CoA carboxylase, and methylcrotonyl-CoA carboxylase as targets for folate antagonists provides a molecular mechanism underlying some of the adverse effects of this class of drugs. These enzymes are involved in key metabolic pathways, and defects in their expression and/or activity cause deleterious consequences as described above.

The present invention makes use of the finding that folate antagonists bind to glutathione synthase, pyruvate carboxylase, propionyl-CoA carboxylase, biotin carboxylase, acetyl-CoA carboxylase, and methylcrotonyl-CoA carboxylase to provide methods and compositions for the improved treatment of neoplastic, hyperproliferative, and immune disorders. For example, the present invention encompasses methods of screening for folate antagonists having increased binding selectivity for one or more folate-dependent enzymes. Compounds that inhibit one or more folate-dependent enzymes but have a reduced binding affinity for one or more enzymes selected from glutathione synthase, pyruvate carboxylase, propionyl-CoA carboxylase, biotin carboxylase, acetyl-CoA carboxylase, and methylcrotonyl-CoA carboxylase will have a decreased risk of adverse effects when used in treatment.

The finding that g glutathione synthase, pyruvate carboxylase, propionyl-CoA carboxylase, biotin carboxylase, acetyl-CoA carboxylase, and methylcrotonyl-CoA carboxylase are targets of folate antagonists also provides biomarkers useful in predicting whether a subject will have an increased risk of adverse effects as a result of treatment with a folate antagonist, and in selecting a therapy for treating a neoplastic, hyperproliferative, or immune disorder in a subject. Individuals that have a lower level of glutathione synthase, pyruvate carboxylase, propionyl-CoA carboxylase, biotin carboxylase, acetyl-CoA carboxylase or methylcrotonyl-CoA carboxylase expression or activity will be more sensitive to the inhibition of this enzyme by folate antagonists. Accordingly, the present invention provides methods of predicting a subjects risk for adverse effects from treatment with a folate antagonist by determining the level of glutathione synthase, pyruvate carboxylase, propionyl-CoA carboxylase, biotin carboxylase, acetyl-CoA carboxylase or methylcrotonyl-CoA carboxylase in the subject by analyzing a sample from the subject, and for selecting a therapy for a subject based on their risk for adverse effects.

I. Methods of Screening for Improved Folate Antagonists

In one embodiment, the present invention provides a method of screening compounds to identify a folate antagonist having increased selectivity. By a “folate antagonist,” it is intended a compound that inhibits the activity of at least one folate-dependent enzyme. By a “folate-dependent enzyme,” it is intended an enzyme which requires folate or a folate metabolite to perform at least one of its catalytic activities. In some embodiments, the folate antagonist inhibits the activity of at least one folate-dependent enzyme selected from dihydrofolate reductase (EC 1.5.1.3), folylpolyglutamate synthetase (EC 6.3.2.17), glycinamide ribonucleotide formyltransferase (EC 2.1.2.2), aminoimidzole carboxamide ribonucleotide formyltransferase (EC 5,3,1,16), and thymidylate synthase (EC 2.1.1.45). The “selectivity” of a folate antagonist as used herein refers to the degree to which the folate antagonist produces the desired effect of alleviating at least one symptom associated with a neoplastic, hyperproliferative, or immune disorder in relation to the adverse effects of the folate antagonist.

In some embodiments, the identified folate antagonists have improved binding selectivity for a folate-dependent enzyme such as, for example, dihydrofolate reductase, folylpolyglutamate synthetase, glycinamide ribonucleotide formyltransferase, aminoimidzole carboxamide ribonucleotide formyltransferase, and thymidylate synthase. By “binding selectivity” it is intended the degree to which the folate antagonist binds the folate-dependent enzyme relative to other proteins in cell, particularly glutathione synthase, pyruvate carboxylase, propionyl-CoA carboxylase, biotin carboxylase, acetyl-CoA carboxylase, and methylcrotonyl-CoA carboxylase.

The methods comprise identifying one or more compounds that inhibit the activity of a folate-dependent enzyme (i.e. a folate antagonist) and screening one or more of the identified folate antagonists to determine the level of binding of the folate antagonist to at least one enzyme selected from glutathione synthase, pyruvate carboxylase, propionyl-CoA carboxylase, biotin carboxylase, acetyl-CoA carboxylase, and methylcrotonyl-CoA carboxylase. A folate antagonist that has a low level of binding to one or more enzymes selected from glutathione synthase, pyruvate carboxylase, propionyl-CoA carboxylase, biotin carboxylase, acetyl-CoA carboxylase, and methylcrotonyl-CoA carboxylase is identified as a folate antagonist having increased selectivity. A “low level of binding” to glutathione synthase, pyruvate carboxylase, propionyl-CoA carboxylase, biotin carboxylase, acetyl-CoA carboxylase or methylcrotonyl-CoA carboxylase according to the invention is a level of binding that is lower than that observed under the same binding conditions for methotrexate. The level of binding of the folate antagonists may be determined for at least one, at least two, or all three enzymes selected from glutathione synthase, pyruvate carboxylase, propionyl-CoA carboxylase, biotin carboxylase, acetyl-CoA carboxylase, and methylcrotonyl-CoA carboxylase. In some embodiments, the binding affinity of the folate antagonist having increased selectivity is at least 5 times greater, at least 10 times greater, at least 20 times greater, at least 50 times greater, at least 100 times greater, at least 500 times greater, at least 10³ times greater, or at least 10⁴ greater for a folate-dependent enzyme than for at least one enzyme selected from glutathione synthase, pyruvate carboxylase, propionyl-CoA carboxylase, biotin carboxylase, acetyl-CoA carboxylase, and methylcrotonyl-CoA carboxylase. In some embodiments, the folate antagonist having increased selectivity has a significantly decreased risk of adverse effects in treatment.

The present invention also provides methods of comparing folate antagonists to identify those having the greatest selectivity and/or the greatest binding selectivity. The method comprises screening two or more folate antagonists to determine their level of binding to one or more enzymes selected from glutathione synthase, pyruvate carboxylase, propionyl-CoA carboxylase, biotin carboxylase, acetyl-CoA carboxylase, and methylcrotonyl-CoA carboxylase, and selecting the folate antagonist that has the lowest level of binding to one or more of these enzymes as the folate antagonist having the greatest selectivity or binding selectivity. The level of binding of the folate antagonists may be determined for at least one, at least two, or all six enzymes selected from glutathione synthase, pyruvate carboxylase, propionyl-CoA carboxylase, biotin carboxylase, acetyl-CoA carboxylase, and methylcrotonyl-CoA carboxylase. The method may be used to select a folate antagonist for use in therapy for a neoplastic, hyperproliferative, or immune disorder.

The invention also provides a method of identifying a folate antagonist having a reduced risk of causing an adverse effect when it is used for therapy in a subject. By an “adverse effect” of a therapy, it is intended any change in the physiologic state of the patient caused by the therapy that does not contribute to the therapeutic efficacy (i.e. the ability of the therapy to alleviate at least one of the symptoms associated with the condition to be treated) of the therapy. A folate antagonist-associated adverse effect according to the invention may be any disorder correlated with the use of folate antagonists, including, for example, alopecia, dermatitis, interstitial pneumonitis, nephrotoxicity, defective oogenesis or spermatogenesis, abortion, teratogenesis, hepatotoxicity, hepatic dysfunction, cirrhosis, central nervous system damage, myelosuppression, mucositis, transaminitis, leukopenia, neutropenia, and thrombocytopenia.

The methods involve screening two or more folate antagonists to determine their level of binding to glutathione synthase, pyruvate carboxylase, propionyl-CoA carboxylase, biotin carboxylase, acetyl-CoA carboxylase, and/or methylcrotonyl-CoA carboxylase. The folate antagonist that has the lowest level of binding to one or more of these enzymes is identified as the folate antagonist having a reduced risk of causing an adverse effect when it is used for therapy in a subject.

The invention further comprises folate antagonists identified or selected according to the methods of the invention, and improved methods of treatment with a folate antagonist in a subject. The improved methods of treatment involve treating the subject with a folate antagonist identified according to the screening methods of the invention.

In some embodiments, the methods of the invention involve determining the level of binding of a compound, such as a folate antagonist, to at least one enzyme selected from glutathione synthase, pyruvate carboxylase, propionyl-CoA carboxylase, biotin carboxylase, acetyl-CoA carboxylase, and methylcrotonyl-CoA carboxylase. The level of binding of a compound to the enzyme is determined by measuring the binding affinity of the compound for the enzyme or by measuring the activity of the enzyme in the presence of the compound.

A. Determining the Level of Binding of a Compound to Glutathione Synthase, Pyruvate Carboxylase, Propionyl-CoA Carboxylase, Biotin Carboxylase, Acetyl-CoA Carboxylase, and/or Methylcrotonyl-CoA Carboxylase by Measuring the Binding Affinity of the Compound for One or More of These Enzymes.

In some embodiments of the methods of the present invention, the level of binding of a compound to glutathione synthase, pyruvate carboxylase, propionyl-CoA carboxylase, biotin carboxylase, acetyl-CoA carboxylase, and/or methylcrotonyl-CoA carboxylase is determined by measuring the binding affinity of the compound for glutathione synthase, pyruvate carboxylase, propionyl-CoA carboxylase, biotin carboxylase, acetyl-CoA carboxylase, and/or methylcrotonyl-CoA carboxylase. The binding affinity is a measure of the strength of the noncovalent binding between the compound and glutathione synthase, pyruvate carboxylase, propionyl-CoA carboxylase, biotin carboxylase, acetyl-CoA carboxylase, and/or methylcrotonyl-CoA carboxylase and may be determined by measuring the amount of the compound that binds to glutathione synthase, pyruvate carboxylase, propionyl-CoA carboxylase, biotin carboxylase, acetyl-CoA carboxylase, and/or methylcrotonyl-CoA carboxylase in comparison to the amount of the compound that does not bind to the enzyme. In some embodiments, the binding affinity of the compound for the enzyme is determined by calculating an affinity constant (equilibrium constant). In other embodiments, where the binding affinity of two or more folate antagonist is to be compared, it may be sufficient to determine the relative binding affinity of the inhibitors for glutathione synthase, pyruvate carboxylase, propionyl-CoA carboxylase, biotin carboxylase, acetyl-CoA carboxylase, and/or methylcrotonyl-CoA carboxylase.

Any method known in the art may be used to determine the amount of the compound that binds to glutathione synthase, pyruvate carboxylase, propionyl-CoA carboxylase, biotin carboxylase, acetyl-CoA carboxylase, and/or methylcrotonyl-CoA carboxylase under a given set of conditions. Both direct binding assays and competitive binding assays can be used in a variety of different formats. For a description of different formats for binding assays, including competitive binding assays and direct binding assays, see, for example, Seethala and Femades, eds. (2001) Handbook of Drug Screening (Marcel Dekker, New York); and Mei and Czarnik, eds. (2002) Integrated Drug Discovery Technologies (Marcel Dekker, New York), both of which are herein incorporated by reference in their entirety for all purposes.

In some embodiments, the binding assays employ labeled components. The label can be in a variety of forms. The label may be coupled directly or indirectly to the desired component of the assay according to methods well known in the art. A wide variety of labels may be used. The component may be labeled by any one of several methods. In some embodiments, a radioactive label incorporating ³H, ¹²⁵I, ³⁵S, ¹⁴C, or ³²P is used. Alternatively, non-radioactive labels such as chromophores, fluorophores, chemiluminescent agents, enzymes, and antibodies that can serve as specific binding pair members for a labeled ligand may be used.

In certain embodiments, the binding assay is carried out in an a cell-free assay system, while in other embodiments the binding assay is carried out in a cell that naturally expresses glutathione synthase, pyruvate carboxylase, propionyl-CoA carboxylase, biotin carboxylase, acetyl-CoA carboxylase, and/or methylcrotonyl-CoA carboxylase or has been engineered to express glutathione synthase, pyruvate carboxylase, propionyl-CoA carboxylase, biotin carboxylase, acetyl-CoA carboxylase, and/or methylcrotonyl-CoA carboxylase. Accordingly, the glutathione synthase, pyruvate carboxylase, propionyl-CoA carboxylase, biotin carboxylase, acetyl-CoA carboxylase, and/or methylcrotonyl-CoA carboxylase used in the methods of the invention may be either native or recombinantly produced in a host cell or in vitro.

B. Determining the Level of Binding of a Compound to Glutathione Synthase, Pyruvate Carboxylase, Propionyl-CoA Carboxylase, Biotin Carboxylase, Acetyl-CoA Carboxylase, and/or Methylcrotonyl-CoA Carboxylase by Measuring the Activity of the Enzyme in the Presence of the Compound.

In some embodiments of the invention, the level of binding of a compound to glutathione synthase, pyruvate carboxylase, propionyl-CoA carboxylase, biotin carboxylase, acetyl-CoA carboxylase, and/or methylcrotonyl-CoA carboxylase is determined by measuring the activity of the enzyme in the presence of the compound. Methods for measuring the activity of glutathione synthase, pyruvate carboxylase, propionyl-CoA carboxylase, biotin carboxylase, acetyl-CoA carboxylase, and methylcrotonyl-CoA carboxylase are known in the art. See, for example, Delvin et al. (1974) Biochem. Med. 10:97-106; Takada et al. (1982) J. Inherit. Metab. Dis. 5:69-70; Gali and Board (1995) Biochem. J. 310:353-8; Wexler et al. (1998) Pediatr. Res. 43: 579-84; Chen et al. (2000) Am. J. Physiol. Endorinol. Metab. 279:E1202-06; Dean et al. (2000) Diabetes 49:1295-1300; Van Coster et al. (1998) Prenat. Diagn. 18:1041-4; Holzinger et al. (2001) Hum. Mol. Gen. 10:1299-1306; Kowluru et al. (2001) Diabetes 50:1580-87; Sloane et al. (2001) J. Biol. Chem. 276:24991-96; Clayero et al. (2002) Biochim. Biophys. Acta 1588:119-25; Rathman et al. (2002) J. Nutr. 132:3405-10; and Perez-Cerd et al. (2003) Biochim. Biophys. Acta 1638:43-9; each of which is herein incorporated in its entirety by reference. In some embodiments, a synthetic and/or labeled substrate is used to determine enzyme activity.

In some embodiments, the assays for enzyme activity are performed in a high-throughput format. For a description of enzyme activity assays and assay formats, see, for example, Seethala and Femades, eds. (2001) Handbook of Drug Screening (Marcel Dekker, New York); and Mei and Czarnik, eds. (2002) Integrated Drug Discovery Technologies (Marcel Dekker, New York), both of which we herein incorporated by reference in their entirety for all purposes.

The activity of glutathione synthase, pyruvate carboxylase, propionyl-CoA carboxylase, biotin carboxylase, acetyl-CoA carboxylase, and/or methylcrotonyl-CoA carboxylase may be determined using a cell-free assay system or a cell-based screening assays. When the activity assay is performed in a cell-free system, the glutathione synthase, pyruvate carboxylase, propionyl-CoA carboxylase, biotin carboxylase, acetyl-CoA carboxylase, and/or methylcrotonyl-CoA carboxylase may be purified from a tissue or recombinantly produced. Glutathione synthase, pyruvate carboxylase, propionyl-CoA carboxylase, biotin carboxylase, acetyl-CoA carboxylase, and methylcrotonyl-CoA carboxylase are expressed in a number of tissues including brain, kidney, lung, and liver, making cells from these tissues a particularly good source of the native enzymes.

In some embodiments, cell-based screening assays utilize cells or cell lines that express native glutathione synthase, pyruvate carboxylase, propionyl-CoA carboxylase, biotin carboxylase, acetyl-CoA carboxylase, and/or methylcrotonyl-CoA carboxylase. In other embodiments, recombinant host cells expressing glutathione synthase, pyruvate carboxylase, propionyl-CoA carboxylase, biotin carboxylase, acetyl-CoA carboxylase, and/or methylcrotonyl-CoA carboxylase are used.

C. Compounds

In some embodiments the methods comprise screening folate antagonists to identify those having increased selectivity. The folate antagonists to be screened may be in the form of therapeutic cocktails, mixtures or combinations of agents. Alternatively, the folate antagonists may be cruder extracts in various stages of purification or characterization, or pure compounds. Any folate antagonists may be used in the methods of the invention. Non-limiting examples are provided, for example in U.S. Pat. Nos. 2,512,572, 3,892,801, 3,989,703, 4,057,548, 4,067,867, 4,079,056, 4,080,325, 4,136,101, 4,306,064, 4,374,987, 4,421,913, 4,767,859, 3,981,983, 4,376,767, 4,401,592, 4,785,080, 4,816,395, 4,983,586, 5,024,998, 5,106,950, 5,166,149, 5,292,731, 5,698,556, as well as DeGraw et al. (1990) J. Med. Chem. 33:673-7; Bartlett et al. (1995) 39:2436-2441; Lehman (2002) Expert Opin. Investig. Drugs 11:1775-87 Graffier-Nordberg (2003), J. Med. Chem. 46:3455-62, and Purcell and Ettinger (2003) Current Oncology Reports 5:114-125; each of which is herein incorporated in its entirety by reference. Thus, the present invention encompasses the use of folate analogs such as methotrexate, trimetrexate, edatrexate, raltitrexed, pemetrexed, ZD9311, lometrexol, GW1843, AD9331, nolatrexed (AG337), and analogs thereof. See, Prucell and Ettinger (2003) Current Oncology Reports 5:114-25.

In an alternative embodiment of the invention, libraries of compounds are screened to identify folate antagonists. Candidate compounds that may be screened to identify selective folate antagonists according to the methods of the invention include, for example, small organic molecules (e.g., molecules obtained from combinatorial and natural product libraries).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; Gallop et al. (1994) J. Med. Chem. 37:1233; and Ghose and Vishwanadhan, eds. (2001) Combinatorial Library Design and Evaluation: Principles, Software Tools, and Applications in Drug Discovery (Marcel Dekker, New York), each of which is herein incorporated by reference in its entirety.

Methods of determining whether a particular compound inhibits the activity of a folate-dependent enzyme are known in the art, and are described, for example, in Cox and Harrnenberg (1992) J. Biochem. Biophys Methods 25:17-23; Bullerjahn and Freisheim (1992) J. Biol. Chem. 267:864-870; Hughey et al. (1993) Mol. Pharmacol. 44:316-23; Caperelli and Giroux (1997) Arch. Biochem. Biophys. 341:98-103; Poch et al. (1998) Protein Expr. Purif. 12:17-24; Ehrnrooth et al. (2000) Clin. Chim. Acta. 290:129-44; and Wu and Dolnick (2003) Mol. Pharmacol. 63:167-173; each of which is herein incorporated in its entirety by reference.

II. Methods and Kits for Identifying Subjects having an Increased Risk of Adverse Effects from treatment with Folate Antagonist

The present invention encompasses methods of screening subjects to identify those having an increased risk of adverse effects following treatment with a folate antagonist, and for selecting a therapy for a subject affected by a neoplastic, hyperproliferative, or immune disorder. The methods rely on the finding that methotrexate, the most commonly used folate antagonist, binds to glutathione synthase, pyruvate carboxylase, propionyl-CoA carboxylase, biotin carboxylase, acetyl-CoA carboxylase, and methylcrotonyl-CoA carboxylase, and that individuals having lower levels of expression or activity of these enzymes will have increased risk of adverse effects from folate antagonists. Accordingly, glutathione synthase, pyruvate carboxylase, propionyl-CoA carboxylase, biotin carboxylase, acetyl-CoA carboxylase, and methylcrotonyl-CoA carboxylase serve as biomarker for the risk of an adverse effect as a result of treatment with a folate antagonist.

In some embodiments, the methods comprise determining the level of one or more enzymes selected from glutathione synthase, pyruvate carboxylase, propionyl-CoA carboxylase, biotin carboxylase, acetyl-CoA carboxylase, and methylcrotonyl-CoA carboxylase in a sample from a subject and comparing this level with a range of values representing the levels of these enzymes in subjects who have experienced adverse effects as a result of treatment with a folate antagonist. A subject having a level of glutathione synthase, pyruvate carboxylase, propionyl-CoA carboxylase, biotin carboxylase, acetyl-CoA carboxylase, and/or methylcrotonyl-CoA carboxylase, falling within the range of values for the levels of these enzymes in subjects who have experienced adverse effects following treatment with a folate antagonist is identified as a subject having an increased risk of a metabolic disorder following treatment with a folate antagonist.

In other embodiments, the method of screening subjects to identify those having an increased risk of developing adverse effects following treatment with a folate antagonist, the level of at least one enzyme selected from glutathione synthase, pyruvate carboxylase, propionyl-CoA carboxylase, biotin carboxylase, acetyl-CoA carboxylase, and methylcrotonyl-CoA carboxylase in a sample from the subject is compared with a range of values representing the levels of the same enzyme or enzymes in normal subjects. A subject having a level of at least one enzyme selected from glutathione synthase, pyruvate carboxylase, propionyl-CoA carboxylase, biotin carboxylase, acetyl-CoA carboxylase, and methylcrotonyl-CoA carboxylase that is significantly lower than the level in normal subjects is identified as a subject having an increased risk of developing an adverse reaction following treatment with a folate antagonist. By an expression level that is “significantly lower” than the level in normal subjects, it is intended an expression level that is statistically significantly lower than that seen in normal subjects. A statistical test may be used to test whether a change in expression level measured for a gene after treatment is more likely to result from an actual change in the expression of the gene rather than from any variability present in the experimental system.

The level of glutathione synthase, pyruvate carboxylase, propionyl-CoA carboxylase, biotin carboxylase, acetyl-CoA carboxylase, and/or methylcrotonyl-CoA carboxylase measured may be either the level of expression of the enzyme or the level of activity of the enzyme. The level of may be determined for at least one, at least two, or all three enzymes selected from glutathione synthase, pyruvate carboxylase, propionyl-CoA carboxylase, biotin carboxylase, acetyl-CoA carboxylase, and methylcrotonyl-CoA carboxylase.

The methods are also useful in selecting a therapy for a subject affected by a neoplastic, hyperproliferative, or immune disorder. When a subject is diagnosed as being at risk for adverse effects of treatment with a folate antagonist as a result of having a low level of glutathione synthase, pyruvate carboxylase, propionyl-CoA carboxylase, biotin carboxylase, acetyl-CoA carboxylase, and/or methylcrotonyl-CoA carboxylase, a therapy can be selected to minimize this risk. For example, the selected therapy may comprise treatment with a class of drugs other than folate antagonists. Alternatively, the selected therapy may comprise treatment with a folate antagonist having a lower binding affinity for glutathione synthase, pyruvate carboxylase, propionyl-CoA carboxylase, biotin carboxylase, acetyl-CoA carboxylase, and/or methylcrotonyl-CoA carboxylase.

A. Methods for Determining the Expression Level of Glutathione Synthase, Pyruvate Carboxylase, and/or Propionyl-CoA Carboxylase in a Subject

In some embodiments, the methods of predicting whether a subject has an increased risk of adverse effects as a result of treatment with a folate antagonist comprise the step of measuring the expression level of at least one enzyme selected from glutathione synthase, pyruvate carboxylase, PCCA, PCCB, ACC1, ACC2, COA1, COA2, MCCA, and MCCB in the subject. As used herein, an “expression level” or “level of expression” is a value that corresponds to a measurement of the abundance of a gene expression product. Such values may include measurements of RNA levels or protein abundance. Thus, an expression level can be a value that reflects the transcriptional state or the translation state of a gene.

The samples used to determine levels of glutathione synthase, pyruvate carboxylase, propionyl-CoA carboxylase, biotin carboxylase, acetyl-CoA carboxylase, and/or methylcrotonyl-CoA carboxylase in a subject can be derived from a variety of sources including, but not limited to, cells, tissues, blood, urine, or other bodily fluids. When cells or tissues are used, the tissue or cell source may include a tissue biopsy sample, a cell sorted population, cell culture, or a single cell. In some embodiments, the samples of the invention are derived from a human subject, while in other embodiments, the samples are derived from a model organism useful for studying therapy for neoplastic, hyperproliferative, and immune disorders. Examples of such model organisms include, but are not limited to, mammalian model organisms including primate and rodent model systems.

In some embodiments, the samples are taken from the subject prior to treatment with the folate antagonist, while in other embodiments the sample is taken during the course of treatment with the folate antagonist. Samples taken during the course of treatment allow for the monitoring of the effects of the treatment on glutathione synthase, pyruvate carboxylase, propionyl-CoA carboxylase, biotin carboxylase, acetyl-CoA carboxylase, and/or methylcrotonyl-CoA carboxylase activity.

In some embodiments of the invention, a value representing the level of glutathione synthase, pyruvate carboxylase, propionyl-CoA carboxylase, biotin carboxylase, acetyl-CoA carboxylase, and/or methylcrotonyl-CoA carboxylase in a subject is compared to a range of values representing the level of the same enzyme or enzymes in subjects that have experienced an adverse effect following treatment with a folate antagonist. In other embodiments, the value representing the level in the patient is compared with a range of values in normal subjects. Values may be compared for one, two, or all three of the enzymes selected from glutathione synthase, pyruvate carboxylase, propionyl-CoA carboxylase, biotin carboxylase, acetyl-CoA carboxylase, and methylcrotonyl-CoA carboxylase. In this aspect, a subject whose level of glutathione synthase, pyruvate carboxylase, propionyl-CoA carboxylase, biotin carboxylase, acetyl-CoA carboxylase, and/or methylcrotonyl-CoA carboxylase falls within the range of values for the level of the same enzyme or enzymes in subjects who developed adverse effects following treatment with a folate antagonist is identified as a subject at increased risk for a folate antagonist-induced metabolic disorder.

In particular embodiments, the values representing the levels of glutathione synthase, pyruvate carboxylase, propionyl-CoA carboxylase, biotin carboxylase, acetyl-CoA carboxylase, and/or methylcrotonyl-CoA carboxylase in subjects who have experienced an adverse effect following treatment with a folate antagonist are the levels of these enzymes in subjects who have developed adverse effects following treatment with methotrexate

Thus, according to the present invention, the expression of glutathione synthase, pyruvate carboxylase, propionyl-CoA carboxylase, biotin carboxylase, acetyl-CoA carboxylase, and/or methylcrotonyl-CoA carboxylase is correlated with an increased risk of folate antagonist-induced adverse effects. The significance of the correlation between the expression level of g glutathione synthase, pyruvate carboxylase, propionyl-CoA carboxylase, biotin carboxylase, acetyl-CoA carboxylase, and/or methylcrotonyl-CoA carboxylase and an increased prevalence of a adverse effect in response to treatment with a folate antagonist may be determined by a statistical test of significance. Methods for determining the strength of a correlation between the expression level of a differentially-expressed gene and a particular physiologic state are known in the art and are also reviewed in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety.

In one aspect of the invention, the values representing the levels of glutathione synthase, pyruvate carboxylase, propionyl-CoA carboxylase, biotin carboxylase, acetyl-CoA carboxylase, and/or methylcrotonyl-CoA carboxylase in normal subjects or in subjects who have experienced an adverse effect following treatment with a folate antagonist are derived from a population of subjects. The adverse effect may be any adverse effect associated with folate antagonist treatment as described elsewhere herein. By a “population of subjects,” it is intended one or more patients affected by the adverse effect of folate antagonist therapy or one or more normal subjects. The number of patients to be included in the population varies according to the selected therapy. Accordingly, the population of subjects comprises at least one subject, and may also comprise at least two subjects, at least three subjects, at least four subjects, at least five subjects, at least six subjects, at least eight subjects, at least ten subjects, at least fifteen subjects, at least twenty-five subjects, at least fifty subjects, at least one hundred subjects, at least two hundred subjects, and least three hundred subjects, at least five hundred subjects, at least one thousand subjects, or at least ten thousand subjects or more.

The expression levels of these genes may be determined by any method known in the art for assessing the expression level of an RNA or protein molecule in a sample. For example, expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The gene expression monitoring system may also comprise nucleic acid probes in solution.

In one embodiment of the invention, microarrays are used to measure the values to be included in the expression profiles. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, the Examples section. See also, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

In another embodiment, the expression level of glutathione synthase, pyruvate carboxylase, propionyl-CoA carboxylase, biotin carboxylase, acetyl-CoA carboxylase, and/or methylcrotonyl-CoA carboxylase is obtained by measuring the abundance of the glutathione synthase, pyruvate carboxylase, propionyl-CoA carboxylase, biotin carboxylase, acetyl-CoA carboxylase, and/or methylcrotonyl-CoA carboxylase. The abundance of these protein products can be determined, for example, using antibodies specific for these enzymes. The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. The antibody can be coupled to a toxin or imaging agent. A full-length glutathione synthase, pyruvate carboxylase, propionyl-CoA carboxylase, biotin carboxylase, acetyl-CoA carboxylase, and/or methylcrotonyl-CoA carboxylase, or an antigenic peptide fragment of the protein can be used as an immunogen. Preferred epitopes encompassed by the antigenic peptide are regions of the protein that are located on the surface of the protein, e.g., hydrophilic regions, as well as regions with high antigenicity.

Detection of binding by the antibody can be facilitated by coupling it to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

B. Methods for Determining the Activity Level of Glutathione Synthase, Pyruvate Carboxylase, Propionyl-CoA Carboxylase, Biotin Carboxylase, Acetyl-CoA Carboxylase, and/or Methylcrotonyl-CoA Carboxylase in a Subject

In some embodiments, the methods of predicting whether a subject has an increased risk of adverse effects from treatment with a folate antagonist comprise the step of measuring the activity level of glutathione synthase, pyruvate carboxylase, propionyl-CoA carboxylase, biotin carboxylase, acetyl-CoA carboxylase, and/or methylcrotonyl-CoA carboxylase in the subject. Methods for determining the activity of these enzymes are provided elsewhere herein and may be applied to a sample from the subject.

Alternatively, a sample from the patient can be assayed for the presence of particular metabolites that reflect the activity level of glutathione synthase, pyruvate carboxylase, propionyl-CoA carboxylase, biotin carboxylase, acetyl-CoA carboxylase, and/or methylcrotonyl-CoA carboxylase in the subject. For example, in one embodiment, a sample from the patient is assayed to determine the level of a glutathione synthase, pyruvate carboxylase, propionyl-CoA carboxylase, biotin carboxylase, acetyl-CoA carboxylase, and/or methylcrotonyl-CoA carboxylase-related metabolite in the patient.

The samples used to determine the activity level of glutathione synthase, pyruvate carboxylase, propionyl-CoA carboxylase, biotin carboxylase, acetyl-CoA carboxylase, and/or methylcrotonyl-CoA carboxylase in a subject can be derived from a variety of sources including, but not limited to, cells, tissues, blood, urine, or other bodily fluids. When cells or tissues are used, the tissue or cell source may include a tissue biopsy sample, a cell sorted population, cell culture, or a single cell. In some embodiments, the samples of the invention are derived from a human patient, while in other embodiments, the samples are derived from a model organism useful for studying therapy for neoplastic, hyperproliferative, or autoimmune disorders. Examples of such model organisms include, but are not limited to, mammalian model organisms including rodent model systems.

The value representing the activity level of glutathione synthase, pyruvate carboxylase, propionyl-CoA carboxylase, biotin carboxylase, acetyl-CoA carboxylase, and/or methylcrotonyl-CoA carboxylase in the subject is compared to a range of values representing the levels of one or more of these same enzymes in subjects who have experienced an adverse effect following treatment with a folate antagonist as described above for expression levels of glutathione synthase, pyruvate carboxylase, propionyl-CoA carboxylase, biotin carboxylase, acetyl-CoA carboxylase, and methylcrotonyl-CoA carboxylase.

C. Kits

The invention encompasses kits for use in a method of screening subjects to identify those having an increased risk of experiencing an adverse effect following treatment with a folate antagonist, and in a method for selecting a therapy for a subject affected by a neoplastic, hyperproliferative, or immune disorder. The kit includes reagents for use in determining a value representing the level of one or more enzymes selected from glutathione synthase, pyruvate carboxylase, propionyl-CoA carboxylase, biotin carboxylase, acetyl-CoA carboxylase, and/or methylcrotonyl-CoA carboxylase in a sample taken from the subject. Non-limiting examples of such reagents include arrays, nucleic acid probes, and antibodies. The kit also includes instructions for use in the method of determining whether the subject has an increased risk of experiencing an adverse effect in response to treatment with a folate antagonist. In some embodiments, the instructions additionally provide a range of values representing the levels of at least one enzyme selected from glutathione synthase, pyruvate carboxylase, propionyl-CoA carboxylase, biotin carboxylase, acetyl-CoA carboxylase, and/or methylcrotonyl-CoA carboxylase in subjects who have experienced an adverse effect in response to treatment with a folate antagonist.

Experimental

Molecular targets for the folate antagonist methotrexate were identified as follows. Cell or tissue lysates were produced from porcine liver, porcine lidney, HeLa cells, and whole mice. This lysate was run over an ATP-Sepharose™ column in order to extract all purine-binding proteins. See, WO 00/63694 and U.S. patent application Ser. No. 09/958,787, filed Jan. 16, 2002 and Ser. No. 10/762,078 filed Jan. 21, 2004, each of which is incorporated by reference in its entirety. The ATP-Sepharose™ washed several times, and then eluted with methotrexate at concentrations ranging from 10 nM to 500 μM to identify proteins that bound to this compound. The eluted fractions were run on a 1-dimensional SDS polyacrylamide gel. The eluted proteins were then stained with silver stain, cut out of the gel, and identified using matrix-assisted laser deionization/ionization (MALDI). In addition to DBHR and GART, methotrexate also eluted glutathione synthase, pyruvate carboxylase, propionyl-CoA carboxylase, biotin carboxylase, acetyl-CoA carboxylase, and methylcrotonyl-CoA carboxylase indicating that this drug binds to these proteins. To confirm these results, the lysates were applied to methotrexate covalently linked to agarose and eluted from the methotrexate-agarose with methotrexate. The same proteins that were eluted from ATP-Sepharose™ with methotrexate were also eluted from methotrexate agarose with methotrexate, confirming that these proteins were molecular targets for methotrexate. The experiment was also repeated using folate to elute the purine binding proteome from the ATP-Sepharose column. When folate was used, DHFR, but not glutathione synthase, pyruvate carboxylase, propionyl-CoA carboxylase, biotin carboxylase, acetyl-CoA carboxylase, or methylcrotonyl-CoA carboxylase was eluted.

The finding that the folate antagonist methotrexate binds to glutathione synthase, pyruvate carboxylase, propionyl-CoA carboxylase, biotin carboxylase, acetyl-CoA carboxylase, and methylcrotonyl-CoA carboxylase provides a basis for some of the adverse effects caused by methotrexate treatment. Furthermore, the fact that folate selectively elutes DHFR but not glutathione synthase, pyruvate carboxylase, propionyl-CoA carboxylase, biotin carboxylase, acetyl-CoA carboxylase, and methylcrotonyl-CoA carboxylase from ATP-Sepharose™ demonstrates that it will be possible to identify compounds that bind selectively to DHFR but not to glutathione synthase, pyruvate carboxylase, propionyl-CoA carboxylase, biotin carboxylase, acetyl-CoA carboxylase, and methylcrotonyl-CoA carboxylase.

Various publications, patent applications and patents are cited herein, the disclosures of which are incorporated by reference in their entireties.

That which is claimed:

1. A method of screening compounds to identify a folate antagonist having increased selectivity, said method comprising the steps of:
(a) screening compounds to identify one or more compounds of interest that inhibit the activity of a folate-dependent enzyme; and
(b) screening one or more of the compounds of interest identified in step (a) to determine the level of binding of the compound of interest to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase;
wherein a compound of interest that has a low level of binding to at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase is identified as a folate antagonist having increased selectivity.

2. The method of claim 1 wherein the level of binding of the compound of interest for at least one of said one or more enzymes is determined by measuring the binding affinity of the compound of interest for the enzyme.

3. The method of claim 1 wherein the level of binding of the compound of interest to at least one of said one or more enzymes is determined by measuring the activity of the enzyme in the presence of the compound of interest.

4. The method of claim 3 wherein the activity of the enzyme is measured in a cell-based assay.

5. The method of claim 3 wherein the activity of the enzyme is measured in a cell-free assay.

6. The method of claim 5, wherein the enzyme is recombinantly produced.

7. A folate antagonist identified by the method of claim 1.

8. A method of screening for a selective folate antagonist, said method comprising:
(a) screening two or more folate antagonists to determine their level of binding to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase; and
(b) selecting the folate antagonist that has the lowest level of binding to at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase as a selective folate antagonist.

9. The method of claim 8 wherein the level of binding of the folate antagonist for at least one of said one or more enzymes is determined by measuring the binding affinity of the folate antagonist for the enzyme.

10. The method of claim 8 wherein the level of binding of the folate antagonist to at least one of said one or more enzymes is determined by measuring the activity of the enzyme in the presence of the compound of interest.

11. The method of claim 10 wherein the activity of the enzyme is measured in a cell-based assay.

12. The method of claim 10 wherein the activity of the enzyme is measured in a cell-free assay.

13. A folate antagonist having increased selectivity identified by the method of claim 8.

14. A method of selecting a folate antagonist for use in therapy for a neoplastic, hyperproliferative, or immune disorder, said method comprising:
(a) screening two or more folate antagonists to determine their level of binding to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase; and
(b) selecting the folate antagonist that has the lowest level of binding to at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase as the folate antagonist for use in therapy for neoplastic, hyperproliferative, or immune disorders.

15. A method of identifying a folate antagonist having a reduced risk of causing adverse effects in a subject, said method comprising:
(a) screening two or more folate antagonists to determine their level of binding to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase; and
(b) identifying the folate antagonist that has the lowest level of binding to at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase; as a folate antagonist having a reduced risk of causing adverse effects in a subject.

16. A method for determining the selectivity of a folate antagonist, said method comprising determining the binding affinity of the folate antagonist for one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase.

17. A method for predicting whether a folate antagonist will have adverse effects in treatment, said method comprising determining the level of binding of the folate antagonist to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase.

18. In a method of screening for folate antagonists for use in the treatment of neoplastic, hyperproliferative, and immune disorders, an improvement comprising determining the level of binding of one or more folate antagonists to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase.

19. In a method of screening for folate antagonists having increased selectivity, an improvement comprising determining the level of binding of one or more folate antagonists to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase.

20. A method of screening for drugs that may be used to improve treatment of a neoplastic, hyperproliferative, or immune disorder, said method comprising:
(a) identifying one or more compounds of interest that inhibit the activity of a folate-dependent enzyme to thereby identify a folate antagonist; and
(b) screening one or more of the folate antagonists identified in step (a) to determine their level of binding to one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase;
wherein a folate antagonist that has a low level of binding to at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase is identified as a drug that may be used to improve treatment of a neoplastic, hyperproliferative, or immune disorder.

21. In a method of treating a neoplastic, hyperproliferative, or immune disorder in a subject, an improvement comprising treating the subject with a folate antagonist identified by the method of claim 1.

22. A method of determining whether a subject has an increased risk of an adverse effect as a result of treatment with a folate antagonist, said method comprising the steps of
(a) determining a value representing the level of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase, in a sample taken from the subject; and
(b) comparing the value measured in step (a) with a range of values representing the level of the same enzyme in subjects who have experienced adverse effects as a result of treatment with a folate antagonist;
wherein a subject having a level of at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase falling within the range of values representing the level of the same enzyme in subjects who have experienced adverse effects as a result of treatment with a folate antagonist is identified as a subject having an increased risk of adverse effects following treatment with a folate antagonist.

23. The method of claim 22, wherein the value measured in step (a) represents the expression level one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase in the subject.

24. The method of claim 22, wherein the value measured in step (a) represents the activity level of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase in the subject.

25. The method of claim 22, wherein said sample is blood.

26. The method of claim 22, wherein said sample is urine.

27. The method of claim 22, wherein the values representing the level of the enzyme in subjects who have experienced adverse effects as a result of treatment with a folate antagonist are the levels of the enzyme in subjects who have developed an adverse effect as a result of treatment with methotrexate.

28. The method of claim 22, wherein the sample is taken from the subject before treatment with the folate antagonist.

29. A kit for use in the method of claim 22, said kit comprising reagents for use in determining a value representing the level of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase in a sample taken from the subject and instructions for use in a method of determining whether the subject has an increased risk of adverse effects following treatment with a folate antagonist.

30. The kit of claim 29, wherein said instructions provide a range of values representing the levels of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase in subjects who have experienced adverse effects as a result of treatment with a folate antagonist.

31. The method of claim 22, wherein said adverse effect is nephrotoxicity or hepatotoxicity.

32. A method of determining whether a subject has an increased risk of an adverse effect as a result of treatment with a folate antagonist, said method comprising the steps of
(a) determining a value representing the level of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase, in a sample taken from the subject; and
(b) comparing the value measured in step a) with a range of values representing the level of the same enzyme in normal subjects;
wherein a subject having a level of at least one enzyme selected from the group consisting of glutathione synthase, pyruvate carboxylase, propionyl-CoA carboxylase, biotin carboxylase, acetyl-CoA carboxylase, and methylcrotonyl-CoA carboxylase, that is significantly lower that the value representing the level of the same enzyme in normal subjects is identified as a subject having an increased risk of adverse effects following treatment with a folate antagonist.

33. A method of selecting a therapy for a subject affected by a neoplastic, hyperproliferative, or immune disorder, said method comprising
(a) determining a value representing the level of one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase in a sample taken from the subject;
(b) determining whether the value measured in step (a) falls within a range of values representing the levels of the same enzyme in subjects who have experienced adverse effects following treatment with a folate antagonist; and
(c) selecting a therapy for the patient based on the results of step (b);
wherein a therapy that decreases the risk of an adverse effect following treatment with a folate antagonist is selected when the value measured in step (a) falls within a range of values representing the levels of the same enzymes in subjects who have experienced adverse effects following treatment with a folate antagonist.

34. The method of claim 33, wherein the therapy that decreases the risk of an adverse effect following treatment with a folate antagonist comprises the use of a folate antagonist having a low level of binding for one or more enzymes selected from the group consisting of glutathione synthase, pyruvate carboxylase, propionyl-CoA carboxylase, biotin carboxylase, acetyl-CoA carboxylase, and methylcrotonyl-CoA carboxylase.
